(19) United States
(12) Patent Application Publication
(10) Pub. No.: US 2007/0021716 A1
(43) Pub. Date: Jan. 25, 2007

(54) Nozzle device with skin stretching means

(75) Inventor: Torben Strom Hansen, Kobenhavn (DK)

Correspondence Address: NOVO NORDISK, INC.;PATENT DEPARTMENT, 100 COLLEGE ROAD WEST, PRINCETON, NJ, 08540, US

(73) Assignee: Novo Nordisk A/S, Bagsvaerd (DK)

(21) Appl. No.: 11/453,575

(22) Filed: Jun. 15, 2006

Related U.S. Application Data
(63) Continuation of application No. PCT/DK04/00874, filed on Dec. 16, 2004.

(30) Foreign Application Priority Data
Dec. 18, 2003 (DK) PA 2003 01872

(51) Int. Cl. A61M 5/30

(52) U.S. Cl. 604/68

(57) ABSTRACT

The invention relates to a nozzle device adapted for placement against a skin surface of a subject, the nozzle device providing a means for stretching the skin, the nozzle device being suitable for use in jet injection. More specifically, the skin stretching means is arranged circumferentially relative to a nozzle, the skin stretching means having an initial first configuration corresponding to an initial state in which the skin stretching means is adapted to be placed against a skin surface of the subject, the skin stretching means being moveable to a second configuration, wherein movement of the skin stretching means to the second configuration after the skin stretching means has been placed against the skin of the subject results in the skin being stretched relative to the outlet nozzle.

The invention relates to a nozzle device adapted for placement against a skin surface of a subject, the nozzle device providing a tool for stretching the skin. The nozzle device may advantageously be used in a delivery device to improve interaction between the delivery device and a skin surface. For example, the nozzle device may be used in combination with an impulse generating jet injection device.

### BACKGROUND OF THE INVENTION

Subcutaneous and intramuscular delivery of liquid drugs by injection is common in the medical arts. As some medications, such as insulin, must be given frequently by injection to an individual, it is desirable that the injections can be performed easily.

Many patients dislike needle injections due to pain or fear for needles. Further, blood-borne pathogens, such as HIV and hepatitis, can be transmitted to health care workers by accidental needle-sticks. Also, the disposal of used needles is a growing concern. This disposal presents a problem to individuals other than healthcare workers. Children, for example, may find used needles in the trash, putting them at risk of contracting infection. Discarded needles likewise pose a risk to waste disposal workers.

In efforts to minimize the fears and risks associated with needle injections, several types of needle-free jet injectors have been developed. These devices penetrate the skin using a high velocity fluid jet, and deliver medication into the tissue of a patient. In order to accomplish this, a force is exerted on the liquid medication. Jet injectors, in general, contain a fluid drug which has been transferred into a chamber having a small orifice at one end. A drive means, e.g. a ram, is accelerated using either a coil spring or a compressed gas energy source. The ram impacts a plunger, which in turn creates a high pressure impulse within the chamber. This pressure impulse ejects the fluid medicament through the orifice at high velocity, piercing the skin. The energy source continues to apply a force to the plunger, which quickly propels the drug through the opening in the skin, emptying the syringe in a fraction of a second. The drive means may be adapted to provide a two-stage injection, i.e. a first penetrating burst of drug at a high pressure followed by a subsequent delivery of the remaining amount of drug at a lower pressure.

During injection the nozzle should be fixed at the same point relative to the skin. If this is not the case, the jet can cause so called wet shots where none or only a fraction of the dose is delivered through the skin and the desired blood glucose regulation is jeopardised in case of insulin injection. Another consequence of poor fixation can be lacerations of the skin in case the nozzle moves laterally across the skin during injection.

Addressing this problem, U.S. Pat. Nos. 5,911,703 and 6,406,456 each discloses an injector with an integral suction compartment for pulling the skin against the tip of the injection nozzle. As disclosed, the suction compartment functions to create a seal between the skin area and the injector tip without having to compress the skin area and underlying tissue. Further, the use of a suction compartment can prevent lacerations that can be caused when the injector tip moves relative to the skin during an injection. WO 03/000320 discloses a jet injection device in which sealing between the nozzle aperture and the skin is secured by a nozzle having a truncated cone configuration to thereby embed in the skin to form a hydraulic seal.

In view of the above, it is an object of the present invention to provide a nozzle device which can be used in combination with a jet expelling device, and which aids in providing safe and reliable jet injection of a drug. The nozzle device should be small in size, easy to use and capable of being manufactured cost-effectively.

In the alternative, it is a further object to provide a jet injection device that can be modeled similar in function and configuration as a conventional pen type injector, to make the patient comfortable with the jet injection device, and so that the jet injection device can easily be utilized by a non-professional user, e.g. a insulin requiring diabetic.

### DISCLOSURE OF THE INVENTION

In the disclosure of the present invention, embodiments and aspects will be described which will address one or more of the above objects or which will address objects apparent from the below disclosure as well as from the description of exemplary embodiments.

Correspondingly, in a first aspect a jet expelling device is provided comprising a nozzle portion with an outlet nozzle adapted to be arranged against a skin surface of a subject, and skin stretching means arranged circumferentially relative to the outlet nozzle, the skin stretching means having an initial first configuration corresponding to an initial state in which the skin stretching means is adapted to be placed against the skin surface of the subject, the skin stretching means being moveable to a second configuration, wherein movement of the skin stretching means to the second configuration after the skin stretching means has been placed against the skin of the subject results in the skin being stretched relative to the outlet nozzle. The device further comprises impulse generating means for expelling an amount of drug through the outlet nozzle, the impulse generating means being adapted to create a force for injecting liquid drug through the outlet nozzle and into the subject through the skin when the nozzle portion is arranged against the skin of a subject. The device typically comprises a variable-volume impulse chamber associated with the nozzle and on which the impulse generating means acts to empty the chamber. The impulse chamber may e.g. be prefilled, be filled through the nozzle prior to use, or the drug may be transferred to the impulse chamber from a reservoir within the device. Alternatively, a reservoir may serve as an impulse chamber, an impulse applied to the reservoir expelling only a portion of the drug contained in the reservoir.

By engaging and stretching the skin the likelihood that the nozzle moves relative to the skin during injection is reduced. Further, good contact will be provided between the nozzle and the skin just as stretching of the skin will aid in keeping open the injection channel during injection (e.g. through an initially established channel during the first stage of a two-stage injection), the channel subsequently being “closed” as the stretching action removed. Further, by providing an aid which help ensure proper contact between the nozzle and the skin, the compression at the injection site by the user forcing the nozzle too hard against the skin may be reduced to thereby reduce the likelihood of injection through the subcutaneous layer and into muscle tissue, which is often undesirable, e.g. in the case of insulin injection the pharmaco-kinetics will be altered resulting in unpredictable plasma levels of insulin.

In order to stretch the skin, the skin stretching means should be adapted to provide a low degree of slippage between the skin and the skin stretching means during the stretching action. This may be achieved by a number of means, e.g. by suction action, by providing the skin stretching means with relative sharp edges or by adhesive means for engagement with the skin.

Depending on the position of the skin-engaging nozzle portion before, during and after actuation of the skin stretching and stretching means, the skin can be stretched in different ways. For example, when the nozzle portion engages the skin at an early stage, movement of the skin stretching means between the first and second configurations may result in the skin stretching means being displaced proximally relative to the outlet nozzle, thereby stretching the skin “upwardly around” the nozzle portion. If the nozzle portion engages the skin after movement of the skin stretching means between the first and second configurations, the nozzle will engage a radially stretched skin surface. Indeed, a number of combinations are possible, for example, the skin may be stretched both radially and upwardly relative to the outlet nozzle.

In its most basic form, the skin can be stretched between two opposed points, however, in exemplary embodiment the skin stretching means is arranged such that the skin is stretched circumferentially away from the outlet nozzle, i.e. similar to a drum skin. The skin may be stretched circumferentially by a number of discrete skin-engaging members. For example, in a basic form three such elements may be arranged with a spacing of 120 degrees, however, any desirable number of members may be used. The stretching may also be accomplished by a flexible skin stretching means which continuously surrounds the outlet nozzle.

The skin contacting and stretching means may be operated independently after the nozzle device has been placed against the skin, however, in exemplary embodiment the skin stretching means is adapted to be moved between the first and second configurations when the device is pressed against the skin portion with a given force provided by the user. Thus, in an exemplary embodiment the nozzle device comprises a plurality of skin stretching members (e.g. “fingers” or “flaps”) projecting in a distal-radial direction relative to the outlet nozzle and formed to provide a good grip between the members and the skin. When the nozzle device is pressed against the skin, the members will deflect outwardly thereby stretching the skin. The fingers may be inclined at an angle less than 75 degrees, preferably less than 60 degrees and more preferably less than 45 degrees relative to the axis of the nozzle in the initial position, however, the angle will be dependent upon the actual configuration and flexibility of the fingers.

When it is defined that the skin stretching means has a second configuration, this does not mean that such a second configuration necessarily is well defined, i.e. the second configuration and the degree of stretching associated therewith may depend on how the nozzle device is used by a user. For example, when the skin stretching means is forced against the skin with a given force the skin stretching means (e.g. the above-described fingers) may deflect to a certain degree thereby stretching the skin, whereas the skin stretching means may deflect to a higher degree if the a larger force is applied, this resulting in a greater degree of stretching.

However, the second configuration may also be well defined, for example in case the skin stretching means has a well-defined stop-position or e.g. in case the skin stretching means is bi-stable corresponding to the first and second configurations.

Correspondingly, in an exemplary embodiment the skin stretching means comprises a bi-stable member having a generally distally facing surface (i.e. against the skin) circumferentially surrounding the outlet nozzle, the bi-stable member having a distally concave configuration corresponding to the first configuration, and a distally convex configuration corresponding to the second configuration. To engage the skin, adhesive means is arranged corresponding to a peripheral portion of the distal surface, whereby movement of the skin contacting means between the first and second configurations results in the skin contacting means being displaced proximally relative to the outlet nozzle, thereby stretching the skin.

The nozzle and the skin stretching means may be of unitary construction and adapted to be selectively mounted on a jet expelling device, thereby providing a fluid communication between the expelling device and the outlet nozzle. Typically the nozzle portion will comprise a jet outlet nozzle formed therein and terminating at a distal aperture, the outlet nozzle being adapted to create a skin-penetrating jet of a liquid when the aperture is positioned against the skin surface and a liquid is forced through the nozzle at a given pressure. Although reference is made to a single aperture (or nozzle) the nozzle of the invention may comprise any desired number of additional apertures. Further, the nozzle may comprise a pointed hollow needle adapted to penetrate a superficial layer of the skin of a user, thereby aiding the jet of drug to create an opening in the skin from the surface to the subcutaneous space. Such a needle may be relatively short, e.g. 1 mm or less. The nozzle and skin stretching means may be formed integrally with components of a jet expelling system, e.g. a cartridge containing an amount of drug to be injected or in combination with an impulse chamber. The impulse generating means for expelling an amount of drug through the aperture may be configured in any desirable way, for example corresponding to the jet injection devices shown in U.S. Pat. Nos. 5,911,703 and 5,836,911 or US patent applications 2003/0050592 and 2002/0055707.

Alternatively, the nozzle portion and the skin stretching means may be adapted to be releasably coupled to each other. Correspondingly, in a further aspect the invention provides an injection aid adapted to be mounted on an injection nozzle, such an aid corresponding to the above disclosure with the only difference that the nozzle portion has been replaced with means for engaging such a nozzle portion.

The invention further provides a jet expelling device as described above, further comprising a drive assembly for reducing the volume of the impulse chamber with a reduced force relative to the impulse generating assembly when a portion of the drug has been expelled by the impulse generating assembly. The device may comprise a dose setter for selectable setting a dose of drug to be expelled. The selected amount may be transfered to the impulse chamber from a reservoir provided in the device.

In a further embodiment, the invention provides a jet expelling device of the above-described type, further comprising a dose setter for selectable setting a dose of drug to be expelled and transfer that amount of drug from a reservoir to the impulse chamber, an actuator for actuating the impulse generating assembly and the drive assembly, and an actuatable release, wherein actuation of the release causes the impulse generating assembly to expel a portion of the set dose from the impulse chamber at a high pressure through the outlet nozzle, followed by subsequent expelling of the remaining portion of the set dose from the impulse chamber through the outlet nozzle by means of the drive assembly.

The invention also provides a method of introducing an amount of a drug through the skin of a subject, comprising the steps of (a) providing a jet expelling device comprising a nozzle (e.g. of a type as described above), (b) stretching a skin portion of the subject circumferentially relative to a desired skin location for delivery of the amount of a drug, (c) arranging the nozzle against the desired skin location, and (d) activating the jet expelling device to generate an impulse for expelling an amount of drug through the nozzle and thereby through the stretched skin portion. Skin stretching means (e.g. of a type as described above) may be associated with the nozzle, whereby the skin portion is stretched when the nozzle is arranged against the desired skin location.

As used herein, the term “drug” is meant to encompass any drug-containing flowable medicine or medicament capable of being passed through a nozzle under high pressure in a controlled manner, such as a liquid, solution, gel or fine suspension. Representative drugs include pharmaceuticals such as peptides, proteins, and hormones, biologically derived or active agents, hormonal and gene based agents, nutritional formulas and other substances in both solid (dispensed) or liquid form. In the description of the exemplary embodiments reference will be made to the use of insulin.

### BRIEF DESCRIPTION OF THE DRAWINGS

In the following the invention will be further described with references to the drawings, wherein

FIG. 1 shows a perspective view of a nozzle device,

FIG. 2 shows a sectional view of a nozzle device in an initial configuration,

FIG. 3 shows a sectional view of the nozzle device of FIG. 2 in a second configuration,

FIG. 4 shows a perspective view of a further nozzle device,

FIG. 5 shows a sectional view of a nozzle device in an initial configuration,

FIG. 6 shows a sectional view of the nozzle device of FIG. 5 in a second configuration,

FIG. 7 shows a jet expelling assembly in a sectional view,

FIG. 8 shows the exterior of a further jet expelling assembly,

FIG. 9 shows a further jet expelling assembly in a sectional view, and

FIG. 10 shows an impulse chamber assembly in a sectional view.

In the figures like structures are generally identified by like reference numerals.

### DESCRIPTION OF EXEMPLARY EMBODIMENTS

When in the following terms as “distal”, “proximal” and “radial” or similar relative expressions are used, these only refer to the appended figures and not necessarily to an actual situation of use. The shown figures are schematic representations for which reason the configuration of the different structures as well as there relative dimensions are intended to serve illustrative purposes only.

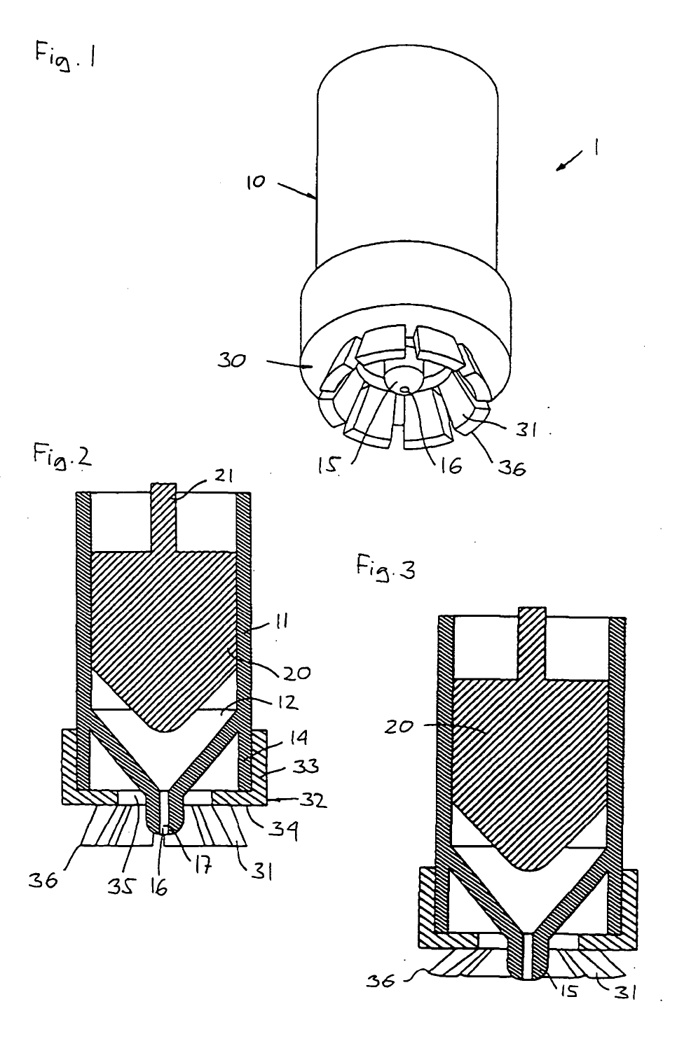

FIG. 1 shows a perspective view of a nozzle device 1 comprising an impulse chamber unit 10 and a thereto connected injection aid in the form of a skin stretching unit 30. The impulse chamber unit comprises a distally facing nozzle portion 15 (in the following also just “nozzle”) with a distal aperture 16 forming an outlet nozzle, and the skin stretching unit comprises a plurality of skin engaging finger members 26 arranged circumferentially around the nozzle portion and projecting in a distal radial direction.

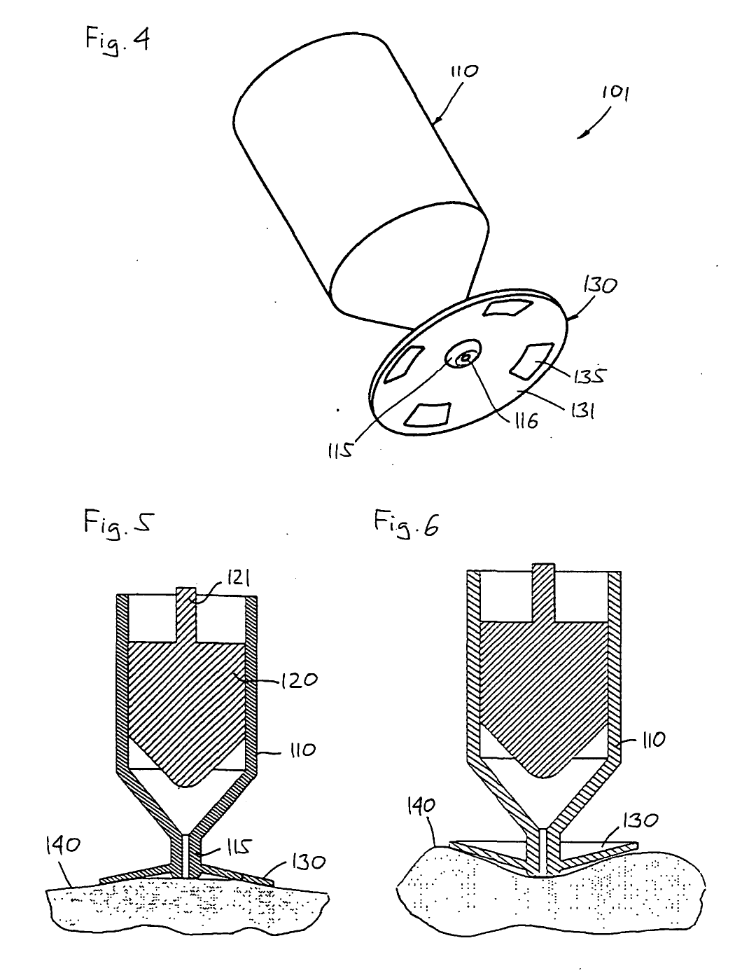

As shown in FIGS. 2 and 3, the impulse chamber unit comprises a housing member 11 in which a piston 20 is slidingly arranged thereby defining a variable-volume impulse chamber 12 in flow communication with the aperture through a nozzle conduit 17. In the shown embodiment the impulse chamber is adapted for being filled with a liquid drug by suction through the nozzle conduit by moving the piston proximally (e.g. by means of a jet injection device engaging a proximal piston extension 21), however the impulse chamber unit may also be provided with an opening in either the housing or the piston (see FIG. 10) allowing a drug to be introduced therethrough by either suction or external pressure in which case the nozzle aperture should be closable. The housing member further comprises a distally extending circumferential skirt portion 14 adapted to engage the skin stretching unit.

The skin stretching unit comprises a body portion 32 having a proximal cylindrical extension 33 adapted to engage the housing skirt and a distally facing surface 34 with an opening 35 through which the nozzle portion projects and from which the skin engaging finger members 31 project. The finger members are provided with relative sharp outer distal edges 36 and are flexible allowing them to deflect in a proximal-radial direction when the fingers are forced against a skin surface as will be explained with reference to FIGS. 2 and 3.

More specifically, FIG. 2 shows a nozzle device connected to a jet injection device (not shown) and containing a volume of drug (not shown) in the impulse chamber, the skin engaging fingers being in an initial non-deflected configuration corresponding to a situation of use in which the nozzle has not yet been forced against the skin of a subject or has just been placed against the skin (not shown) with only minimal pressure. As appears, in the initial state the skin engaging fingers project distally relative to the nozzle. As the nozzle device is forced against the skin, the outer edges engage the skin and as the flexible fingers deflect in a radial direction the skin is correspondingly stretched circumferentially away from the nozzle. FIG. 3 shows the nozzle device in a final “ready-to-inject” configuration in which the skin has been stretched to an intended degree and the nozzle has been forced into engagement with the stretched skin. As appears, the nozzle now projects distally relative to the deflected fingers. The actual position of the nozzle relative to the fingers in the initial and final positions may vary according to the intended use, e.g. the injection parameters and the desired skin location. If desirable, the injection device in combination with which the nozzle device is to be used may be provided with means for detecting the pressure exerted on the skin (e.g. by a pressure sensor arranged between the impulse chamber unit and the jet injection device), thereby indicating to the user that the necessary pressure for asserting that proper stretching of the skin and proper contact between the nozzle and the skin have been reached.

In the shown embodiment the nozzle device comprises two units which may either be permanently attached to each other (e.g. bonded to each other during manufacture) or which may be provided as two separate units which are then assembled by the user. Alternatively, the nozzle device may be manufactured as an integral unit, e.g. with the fingers formed integrally with the housing member.

FIG. 4 shows a perspective view of a further embodiment of a nozzle device 101 comprising an impulse chamber portion 110 with a distally facing nozzle 115 having a distal aperture 116, and a thereto connected skin stretching injection aid in the form of a disc portion 130 arranged circumferentially relative to the nozzle and extending generally in a plane perpendicular to the axial orientation of the nozzle. As seen in FIG. 5 the impulse chamber portion has the same general configuration as the impulse chamber unit of the first embodiment.

The disc portion is in the form of a flexible bi-stable member formed integrally with the impulse chamber portion and having a generally distally facing surface 131 circumferentially surrounding the nozzle, the bi-stable member having a distally concave configuration corresponding to an initial configuration (as seen in FIG. 5), and a distally convex configuration corresponding to a second configuration, the disc being moveable between the two configurations in a “flip-flop” manner in accordance with its bi-stable properties. At the peripheral portion the distal surface of the disc comprises adhesive means 135 adapted for engagement with a skin surface. In the shown embodiment four discrete adhesive patches are used, however, a different number having different configuration(s) may be used. When supplied to the user, a peelable release liner will normally cover the adhesive means. To help remove the nozzle device after use, the disc may be provided with a gripping means (e.g. a flexible strip, not shown) allowing a user to easily tear off the disc from the skin. Indeed, adhesive means may also be used on skin stretching means not having a bi-stable configuration, e.g. as in the above-described first embodiment, the adhesive means here providing a non-slipping engagement.

Turning to a situation of use, FIG. 5 shows a nozzle device connected to a jet injection device (not shown) and containing a volume of drug (not shown) in the impulse chamber, the skin engaging disc portion being in an initial distally-deflected configuration corresponding to a situation of use in which the nozzle has just been placed against a skin surface 140 with light pressure, the adhesive means thereby engaging the skin. As appears, in the initial state the peripheral portion of the disc projects distally relative to the nozzle. As the nozzle device is forced further against the skin, the disc is forced proximally (upwardly) until it assumes a planar configuration of unstable equilibrium and in a “snap”-action deflects proximally thereby pulling the skin to which it is adhered upwardly, whereby the skin it stretched relative to the nozzle. As appears, the nozzle will now project distally relative to the upwardly deflected disc as shown in FIG. 6. The actual position of the nozzle relative to the disc in the initial position may vary according to the intended use, e.g. the injection parameters and the desired skin location.

As the nozzle device is attached to the skin surface by adhesive means, it is no longer crucial that the user forces the nozzle against the skin with a certain force, as the nozzle is kept in contact with the stretched skin via the adhesive means. By this arrangement compression of the injection site can be reduced and thereby the likelihood of injection through the subcutaneous layer and into the underlying muscle tissue.

As for the above-described first embodiment, the impulse chamber portion and the disc portion may be supplied as one or two units.

With reference to FIG. 7 a jet expelling assembly 200 will be described. The assembly comprises a housing 210 with an impulse chamber assembly 230, a dose setting assembly 240 and an impulse generating assembly 250. The dose setting assembly comprises a user actuatable dial member 241 rotationally mounted in a proximal portion 212 of the housing, the dial member being in threaded engagement with a plunger 242, such that clockwise turning of the dial member will move the plunger and thereby the impulse piston distally to expel an amount of fluid from the impulse chamber (see below). The plunger is guided to move longitudinally but it not allowed to rotate. The dose setting assembly preferably comprises a mechanism preventing the dial member to be turned anti-clockwise during normal use.

The impulse chamber assembly comprises a chamber portion 231 with a distal fluid outlet nozzle 232, the chamber portion defining a cavity, an impulse piston 233 slidably received in the cavity along a general axis, and skin stretching means in the form of a plurality of fingers 239 of the type described with reference to FIGS. 1-3. The cavity and the piston in combination define a variable-volume impulse chamber 236. In the shown embodiment the nozzle is formed integrally with the chamber portion. For the shown embodiment, the impulse chamber assembly is delivered to the user as a prefilled unit and further comprises a removable closure member (not shown) sealing the nozzle outlet. The chamber portion is releasable connected to the distal end of the housing by means of a snap mechanism or a threaded connection as shown.

The impulse generating assembly 250 comprises a displaceable transfer tube 251, a spring 252, an actuation lever 253, and a release member 254. The transfer tube is a supported to move longitudinally relative to the housing. The spring engages the proximal end of the transfer tube and forces it distally towards the piston. The lever is pivotally connected to the housing and comprises a toothed portion 255 in engagement with a correspondingly toothed portion 256 on the transfer tube. The release member is pivotally connected to the housing and comprises a hook 257 adapted to engage a corresponding hook 258 on the transfer tube.

In a situation of use the user first actuates the impulse generating assembly by pivoting the actuation lever in the distal direction, this resulting in the transfer tube being moved proximally against the force of the spring to an energized position in which it is locked by engagement with the release member. Preferably a coupling (not shown) is provided in the actuation lever allowing the lever to be returned to its initial position after actuation as well as allowing the transfer tube to move distally without moving the lever. The user also resets the dose setting assembly to its initial position with the plunger in a proximal position. A new pre-filled impulse chamber assembly is then mounted to the housing and in case the dose is to be adjusted the user will expel and discard a desired amount from the impulse chamber by rotating the dial member. The nozzle is then placed against a desired skin surface, this action stretching the skin around the nozzle, where after the user releases the release member, this resulting in the transfer tube being moved distally by the spring, this expelling the drug contained in the impulse chamber through the nozzle and thereby through the skin and into the subcutis.

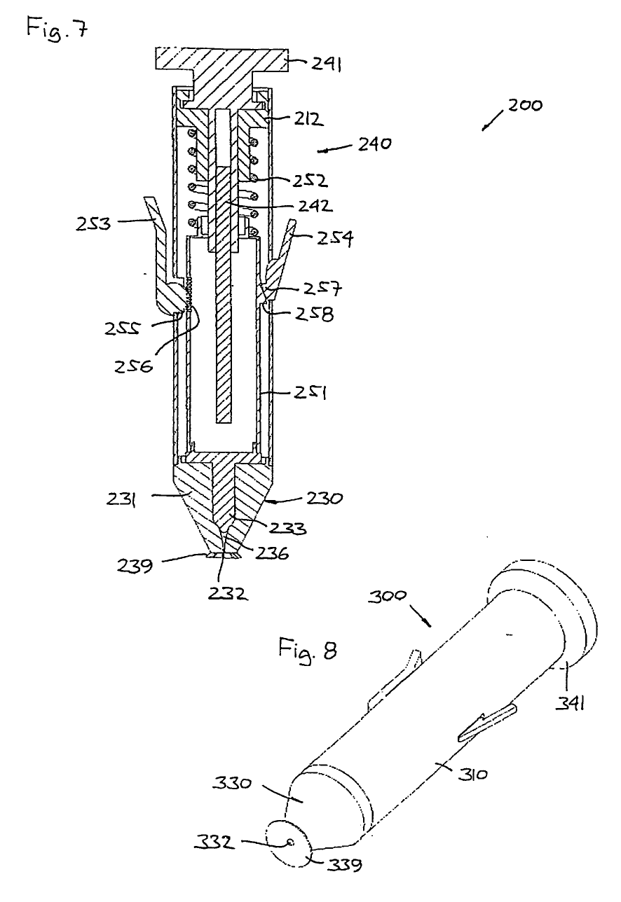

FIG. 8 shows a further jet expelling assembly 300 having the same general construction as the embodiment of FIG. 7, however, in this embodiment the skin stretching means is in the form of a disk shaped member 339 of the type described with reference to FIGS. 4-6.

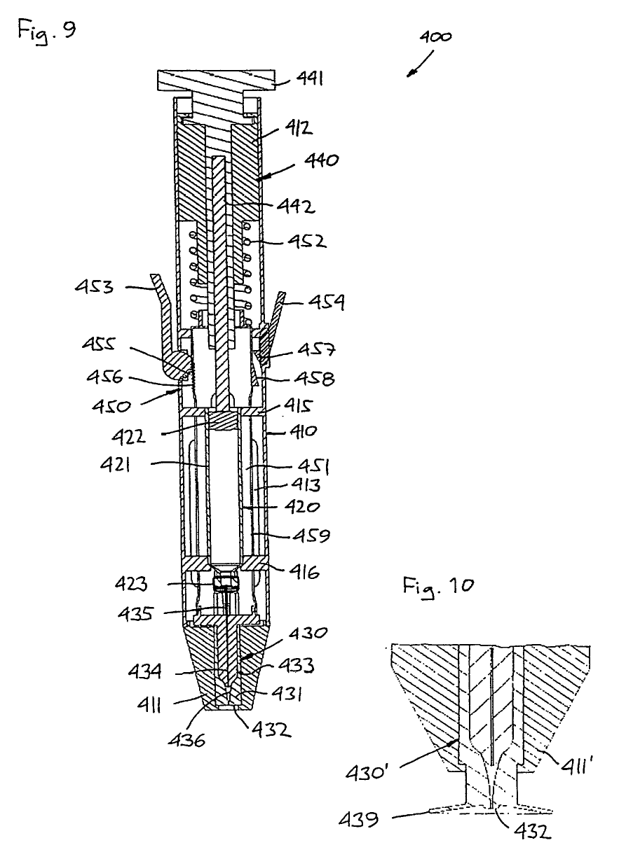

With reference to FIG. 9 a further jet expelling assembly 400 will be described. The assembly comprises a housing 410 in which are arranged a reservoir 420 containing a fluid drug, an impulse chamber assembly 430 in fluid communication with the reservoir, a dose setting assembly 440 and an impulse generating assembly 450. It should be noted that the impulse chamber assembly is shown without skin stretching means (see below). The reservoir is in the form of a columnar cartridge 421 in which a piston 422 is slidably received, the reservoir comprising a distal outlet 423 in the form of a needle-penetratable septum. The reservoir is supported by housing supports 415, 416. The dose setting assembly comprises a user actuatable dial member 441 rotationally mounted in a proximal portion 412 of the housing, the dial member being in threaded engagement with a plunger 442, such that clockwise turning of the dial member will move the plunger and thereby the piston distally to expel an amount of fluid from the reservoir. The dose setting assembly preferably comprises a mechanism preventing the dial member to be turned anti-clockwise during normal use. If the cartridge is replaceable the dose setting assembly will have to be resettable.

The impulse chamber assembly comprises a chamber portion 431 with a distal fluid outlet nozzle 432, the chamber portion defining a cavity, and an impulse piston 433 slidably received in the cavity along a general axis, the piston comprising a through-going channel 434 in fluid communication with a generally straight conduit 435 protruding proximally from the piston and arranged generally in parallel with the general axis. The conduit is adapted to slidably engage the reservoir outlet during relative movement between the piston and the reservoir. The cavity and the piston in combination define a variable-volume impulse chamber 436. In the shown embodiment the nozzle is formed integrally with the chamber portion. When delivered to the user, the impulse chamber further comprises a removable closure member (not shown) sealing the nozzle outlet. The chamber portion is mounted in the housing by means of a mounting member 411 releasable connected to the distal end of the housing, the chamber portion thereby being arranged stationary relative to the reservoir. By this arrangement expelling an amount of drug from the reservoir to the impulse chamber via the conduit causes the piston to move proximally towards the reservoir, the impulse chamber thereby receiving the expelled amount of drug. As appears, the impulse chamber assembly is shown without skin stretching means. Thus reference is made to FIG. 10 showing an impulse chamber assembly 430′ comprising skin stretching means and being adapted to be used with a jet expelling assembly of the type shown in FIG. 9. Indeed, the skin stretching means may have any desirable configuration, e.g. a disk shaped member 439 as shown or flexible finger members of the type shown in FIGS. 1-3. The skin stretching means may also be arranged on the mounting member 411′ or it may be provided as a separate unit to be mounted on either the mounting member or the impulse chamber assembly as shown in FIGS. 1-3.

The impulse generating assembly 450 comprises a displaceable transfer tube 451, a spring 452, an actuation lever 453, and a release member 454. The transfer tube comprises longitudinal side openings 459 allowing it to move longitudinally relative to the housing supports for the reservoir. The spring engages the proximal end of the transfer tube and forces it distally towards the piston. The lever is pivotally connected to the housing and comprises a toothed portion 455 in engagement with a correspondingly toothed portion 456 on the transfer tube. The release member is pivotally connected to the housing and comprises a hook 457 adapted to engage a corresponding hook 458 on the transfer tube. As the housing comprises transparent portions 413 it is possible to inspect the contents of a transparent reservoir through the side openings in the transfer tube.

In a situation of use a new impulse chamber assembly with skin stretching means is mounted in the housing. The user then actuates the impulse generating assembly by pivoting the actuation lever in the distal direction, this resulting in the transfer tube being moved proximally against the force of the spring to an energized position in which it is locked by engagement with the release member. Preferably a coupling (not shown) is provided in the actuation lever allowing the lever to be returned to its initial position after actuation as well as allowing the transfer tube to move distally without moving the lever. Thereafter the user transfers a desired dose of drug from the reservoir to the impulse chamber by rotating the dial member a desired number of increments, this moving the impulse piston proximally as described above. The maximum amount of drug which can be transferred to the impulse chamber is determined by the allowed travel of the impulse piston. In the filled position there should still be a distance between the impulse piston and the transfer tube as the transfer tube should be allowed to accelerate before acting upon the impulse piston to create the desired impulse. Thus a stop mechanism (not shown) may be provided limiting travel of the impulse piston. As appears from FIG. 6 a small amount of air is initially enclosed between the distal end of the piston and the nozzle, however, this amount of air is very small and is not harmful should such an amount of air be injected with the drug. As a final step in preparing the device for injection the user removes the nozzle seal. The nozzle is then placed against a desired skin surface, this action stretching the skin around the nozzle, where after the user releases the release member, this resulting in the transfer tube being moved distally by the spring, this expelling the drug contained in the impulse chamber through the nozzle and thereby through the skin and into the subcutis.

The jet expelling assembly may be a disposable prefilled device as shown, or it may be adapted for used with replaceable cartridges, e.g. by making the distal supports 415 of the housing operatable between an open and a closed position.

The jet expelling assemblies of FIGS. 7 and 9 comprise a single spring providing both an initial impulse to the impulse chamber and the force to empty the impulse chamber once the skin has been penetrated by a jet of drug. Alternatively a jet expelling assembly for injecting fluid medicament into a patient in a two-stage process may be provided. During the first stage fluid is expelled from the injector under relatively high pressure, to create an opening through the skin of the patient. During the second stage, fluid is infused through the opening into the patient at a lower pressure, and for a longer period of time. For example, U.S. Pat. No. 5,911,703, hereby incorporated by reference, discloses a jet expelling assembly with an impulse/drive mechanism including two springs which are positioned to urge against the impulse chamber piston as they elongate. The drive mechanism comprises a transfer rod (i.e. corresponding to the transfer tube of the above-described FIG. 6 embodiment) driven by two coaxially positioned separate springs, which are engaged with the rod. Specifically, the first of the two coaxial springs is an impulse spring which is characterized by a relatively high spring constant and the fact that it is dimensioned to have a relatively short action distance. In comparison with the first spring, the second spring, an injection spring, has a lower spring constant and a longer action distance. Initially, during acceleration of the transfer rod, both the impulse spring and the injection spring push on the rod. However, it is primarily the force of the impulse spring that accelerates the rod. The impulse spring expands until the impulse spring is restrained by a spring stop. After the impulse spring is stopped from expanding, the rod continues moving through a coast distance, until the rod collides with the impulse piston. As a result of this collision, the momentum of the transfer rod causes the piston to accelerate very rapidly. This rapid advancement of the piston is referred to as the impulse stage, and is the first of two stages of advancement of the piston. The impulse stage is very brief, e.g. less than about five milliseconds. Due to the rapid advancement of the piston during the impulse stage, the fluid is expelled through the jet nozzle under high pressure creating a hole or an opening in the skin. After the impulse stage, the injection spring continues to expand and push against the transfer rod. The result is a second stage, referred to as the injection stage. During the injection stage, the injection spring exerts a much smaller force against the rod and piston than the force which was exerted on the piston during the impulse stage. Accordingly, fluid medicament is expelled from the impulse chamber at a much lower pressure and at a much lower rate than during the impulse stage. The duration of the injection stage is much longer than the duration of the impulse stage, and can last as long as five seconds, or longer. During the injection stage, fluid medicament is allowed to slowly infiltrate into the subcutaneous tissue. As appears, such a two-spring two-stage mechanism may be used as an alternative to the one-spring mechanism disclosed in present FIGS. 7 and 9.

In the above description of the preferred embodiments, the different structures and means providing the described functionality for the different components have been described to a degree to which the concept of the present invention will be apparent to the skilled reader. The detailed construction and specification for the different components are considered the object of a normal design procedure performed by the skilled person along the lines set out in the present specification. For example, the distal end of the nozzle may be provided with any desired form securing proper contact between the nozzle and the skin, e.g. rounded (as shown), having the form of a truncated cone or comprising projecting portions engaging the skin to thereby help grip or stretch the skin.

1. A jet expelling device (200, 400) comprising:
a nozzle portion comprising an outlet nozzle (232, 432) adapted to be arranged against a skin surface of a subject,
skin stretching means (239, 439) arranged circumferentially relative to the outlet nozzle, the skin stretching means having an initial first configuration corresponding to an initial state in which the skin stretching means is adapted to be placed against the skin surface of the subject, the skin stretching means being moveable to a second configuration,
wherein movement of the skin stretching means to the second configuration after the skin stretching means has been placed against the skin of the subject results in the skin being stretched relative to the outlet nozzle,
an impulse chamber (236, 436),
an impulse generating assembly (250, 450) for expelling an amount of drug through the outlet nozzle, the impulse generating assembly being adapted to create a force for injecting liquid drug through the outlet nozzle and into the subject through the skin when the nozzle portion is arranged against the skin of a subject.

2. A device as in claim 1, wherein the skin stretching means is adapted for essentially non-slippery engagement with the skin surface.

3. A device as in claim 1, wherein the skin stretching means comprises adhesive means (135) for engagement with the skin.

4. A device as in claim 3, wherein movement of the skin stretching means between the first and second configurations results in the skin stretching means being displaced proximally relative to the outlet nozzle, thereby stretching the skin.

5. A device as in claim 1, wherein movement of the skin stretching means between the first and second configurations results in the skin stretching means being displaced radially relative to the outlet nozzle, thereby stretching the skin.

6. A device as in claim 5, wherein the skin stretching means comprises a plurality of skin stretching members (31) projecting in a distal-radial direction relative to the outlet nozzle.

7. A device as in claim 1, wherein the skin stretching means is bi-stable corresponding to the first and second configurations.

8. A device as in claim 1, wherein the skin is stretched circumferentially away from the outlet nozzle.

9. A device as in claim 3, wherein the skin stretching means comprises a bi-stable member (130) having a generally distally facing surface (131) circumferentially surrounding the outlet nozzle (116), the bi-stable member having a distally concave configuration corresponding to the first configuration, and a distally convex configuration corresponding to the second configuration, the adhesive means being arranged corresponding to a peripheral portion of the distal surface, whereby movement of the skin stretching means between the first and second configurations results in the skin stretching means being displaced proximally relative to the outlet nozzle, thereby stretching the skin.

10. A device as in claim 8, wherein the outlet nozzle projects distally relative to the skin stretching means in the second configuration.

11. A device as in claim 1, wherein the skin stretching means in the first configuration projects distally relative to the outlet nozzle.

12. A device as in claim 1, wherein the skin stretching means is adapted to be moved between the first and second configurations when the device is pressed against the skin portion with a given force.

13. A device as in claim 1, wherein the nozzle portion (10, 15) and the skin stretching means (30) are adapted to be releasably coupled to each other.

14. A device as in claim 1, further comprising a drive assembly for reducing the volume of the impulse chamber with a reduced force relative to the impulse generating assembly when a portion of the drug has been expelled by the impulse generating assembly.

15. A device as in claim 1, further comprising a dose setter (240) for selectable setting a dose of drug to be expelled from the impulse chamber.

16. A device as in claim 1, further comprising a reservoir (421) adapted to contain a fluid drug, and a dose setter (440) for selectable setting a dose of drug to be expelled and transfer that amount of drug from the reservoir to the impulse chamber.

17. A device as in claim 14, further comprising a reservoir and a dose setter for selectable setting a dose of drug to be expelled and transfer that amount of drug from the reservoir to the impulse chamber, an actuator for actuating the impulse generating assembly and the drive assembly, and an actuatable release, wherein actuation of the release causes the impulse generating assembly to expel a portion of the set dose from the impulse chamber at a high pressure through the outlet nozzle, followed by subsequent expelling of the remaining portion of the set dose from the impulse chamber through the outlet nozzle by means of the drive assembly.

18. A method of introducing an amount of a drug through the skin of a subject, comprising the steps:
providing a jet expelling device comprising a nozzle,
stretching a skin portion of the subject circumferentially relative to a desired skin location for delivery of the amount of drug,
arranging the nozzle against the desired skin location, and
activating the jet expelling device to generate an impulse for expelling an amount of drug through the nozzle and thereby through the stretched skin portion.

19. A method as in claim 18, wherein skin stretching means is associated with the nozzle, whereby the skin portion is stretched when the nozzle is arranged against the desired skin location.
